(19) United States
(12) Patent Application Publication
(10) Pub. No.: US 2014/0005482 A1
(43) Pub. Date: Jan. 2, 2014

(54) ENDOSCOPE COOLING APPARATUS

(71) Applicant: OLYMPUS CORPORATION, Tokyo (JP)

(72) Inventor: Jin OHARA, Tokyo (JP)

(73) Assignee: OLYMPUS CORPORATION, Tokyo (JP)

(21) Appl. No.: 14/011,913

(22) Filed: Aug. 28, 2013

Related U.S. Application Data
(63) Continuation of application No. PCT/JP2012/050897, filed on Jan. 18, 2012.

(30) Foreign Application Priority Data
Mar. 24, 2011 (JP) 2011-065475

(51) Int. Cl. A61B 1/12

(52) U.S. Cl. CPC A61B 1/12
USPC 600/158

(57) ABSTRACT

A scope part includes a heat exchanger provided in an end part and having a flow passage for cooling liquid for cooling an functional element, a first tube having a first end connected to a first end of the flow passage of the heat exchanger and extending toward a shaft part, a second tube having a first end connected to a second end of the flow passage of the heat exchanger and extending toward the shaft part, and a displacement-to-flow converting part connected to a second end of the first tube and a second end of the second tube to convert a displacement of a predetermined part to a flow of the cooling liquid. An external device includes a displacement generating part that generates a displacement. The predetermined part of the displacement-to-flow converting part and the displacement generating part are connected by a displacement transmission part.

### CROSS-REFERENCE TO RELATED APPLICATION

The present application is based upon and claims the benefit of priority from the prior Japanese Patent Application No. 2011-065475 filed on Mar. 24, 2011; the entire content of which are incorporated herein by reference.

### TECHNICAL FIELD

The present invention relates to an endoscope cooling apparatus.

### BACKGROUND ART

Conventional endoscope cooling apparatuses have an air/water pumping unit provided in an operation part in order to cool the end of the endoscope, as described in, for example, patent literature 1.

### CITATION LIST

### Patent Literature

Patent Literature 1: Japanese Patent Application Laid-open Publication No. 2009-77762

### SUMMARY OF INVENTION

Technical Problem

In conventional endoscope cooling apparatuses, an air/water pumping unit including a motor or the like is provided in an operation part. This tends to make the operation part large in size and very heavy in weight. The heaviness of the operation part leads to a problem of load on a user who holds the operation part during the operation.

The present invention has been made in view of the above problem, and an object of the present invention is to provide an endoscope cooling apparatus capable of cooling the end of the endoscope without a large increase in the weight of the operation part of the endoscope.

Solution to Problem

To solve the above problem and to achieve the object, an endoscope apparatus according to the present invention has a scope part including an end part, a shaft part, and an operation part to be held, and an external device, and the scope part comprises:
a heat exchanger provided in the end part having a flow passage for a cooling liquid for cooling a functional element that generates heat;
a first tube having a first end connected to a first end of the flow passage of the heat exchanger, and extending toward the shaft part;
a second tube having a first end connected to a second end of the flow passage of the heat exchanger, and extending toward the shaft part; and
a displacement-to-flow converting part connected to a second end of the first tube and a second end of the second tube to convert a displacement of a predetermined part to a flow of the cooling liquid, wherein a displacement generating part is provided in the external device to generate a displacement, and
wherein the predetermined part of the displacement-to-flow converting part and the displacement generating part are connected by a displacement transmission part.

According to a preferred mode of the present invention, it is preferred that the displacement generating part generate a rotational displacement, the displacement transmission part transmit the rotational displacement, and the displacement-to-flow converting part be connected to the second end of the first tube and the second end of the second tube to cause the cooling liquid to circulate in the first tube and the second tube in response to the rotational displacement of the predetermined part.

According to a preferred mode of the present invention, it is preferred that the displacement-to-flow converting part be provided at each of the second end of the first tube and the second end of the second tube, and the cooling liquid move in response to the displacement of the predetermined part.

According to a preferred mode of the present invention, it is preferred that the displacement-to-flow converting part perform an operation of generating a flow of the cooling liquid in the direction from the second end of the first tube to the first end of the first tube and an operation of generating a flow of the cooling liquid in the direction from the second end of the second tube to the first end of the second tube alternately and repeatedly.

According to a preferred mode of the present invention, it is preferred that the displacement generating part provided in the external device comprise a translational displacement generator, and the displacement transmission part cause the predetermined part of at least one displacement-to-flow converting part to displace translationally.

According to a preferred mode of the present invention, it is preferred that the endoscope cooling apparatus comprise:
a first displacement-to-flow converting part provided at the second end of the first tube;
a first translational displacement generator provided in the external device and connected to the first displacement-to-flow converting part by means of the displacement transmission part;
a second displacement-to-flow converting part provided at the second end of the second tube; and
a second translational displacement generator provided in the external device and connected to the second displacement-to-flow converting part by means of the displacement transmission part,
wherein the displacement generating part alternately causes the first translational displacement generator and the second translational displacement generator to generate a pulling force, thereby generating a flow of the cooling liquid.

According to a preferred mode of the present invention, it is preferred that the endoscope cooling apparatus comprise:
a first displacement-to-flow converting part provided at the second end of the first tube;
a first translational displacement generator provided in the external device and connected to the first displacement-to-flow converting part by means of the displacement transmission part;
a second displacement-to-flow converting part provided at the second end of the second tube; and
a biasing spring provided in a predetermined portion of the second displacement-to-flow converting part,
wherein one of forward and backward operations is carried out by a displacement of the first translational displacement generator, and the other of the forward and backward operations is carried out by a displacement caused by a stress of the biasing spring.

According to a preferred mode of the present invention, it is preferred that the displacement-to-flow converting part comprise a chamber having a volumetric capacity varying in response to the displacement of the predetermined part and filled with the cooling liquid.

According to a preferred mode of the present invention, it is preferred that the displacement generating part that generates the translational displacement comprise an actuator that generates a pulling force toward the rear end of the shaft part with respect to the longitudinal direction, the displacement-to-flow converting part comprise a first chamber connected to the actuator and having a volumetric capacity varying in response to the displacement of the predetermined part and a second chamber connected to a biasing spring and having a volumetric capacity varying in response to the displacement of the predetermined part, and the displacement-to-flow converting part cause the cooling liquid to move to and fro in the first tube and the second tube with to-and-fro motion of the actuator along the longitudinal direction of the shaft part.

### ADVANTAGEOUS EFFECTS OF INVENTION

In the endoscope cooling apparatus according to the present invention, the driving part relating to cooling is provided in an external device of the endoscope. Therefore, cooling of the end of the endoscope can be achieved without a large increase in the weight of the operation part of the endoscope. Therefore, the endoscope cooling apparatus according to the present invention is advantageous in reducing a large load due to the weight.

### BRIEF DESCRIPTION OF DRAWINGS

FIG. 1 is a diagram showing the general construction of an endoscope apparatus according to an embodiment of the present invention;

FIG. 2 is a diagram showing the construction of an endoscope according to a first embodiment;

FIG. 3 is a diagram illustrating the connection of the endoscope and a light source device in the first embodiment;

FIG. 4 is a diagram illustrating the flow of cooling liquid in the first embodiment;

FIG. 5 is another diagram illustrating the flow of cooling liquid in the first embodiment;

FIG. 6 is a diagram showing the construction of an endoscope according to a second embodiment;

FIG. 7 is a diagram illustrating the connection of the endoscope and a light source device in the second embodiment;

FIG. 8 is a diagram showing the construction of an endoscope according to a third embodiment;

FIG. 9 is a diagram illustrating the connection of the endoscope and a light source device in the third embodiment;

FIG. 10 is a diagram showing the construction of an endoscope according to a fourth embodiment;

FIG. 11 is a diagram illustrating the connection of the endoscope and a light source device in the fourth embodiment;

FIG. 12 is a diagram showing the construction of an endoscope according to a fifth embodiment;

FIG. 13 is a diagram illustrating the connection of the endoscope and a light source device in the fifth embodiment;

FIG. 14 is a diagram showing the construction of an endoscope according to a sixth embodiment;

FIG. 15 is a diagram illustrating the connection of the endoscope and a light source device in the sixth embodiment;

FIG. 16 is a diagram showing the construction of an endoscope according to a seventh embodiment; and

FIG. 17 is a diagram illustrating the connection of the endoscope and a light source device in the seventh embodiment .

### DESCRIPTION OF EMBODIMENTS

In the following, embodiments of the endoscope cooling apparatus according to the present invention will be described in detail with reference to the drawings. It should be understood that the present invention is by no means limited by the embodiments.

### First Embodiment

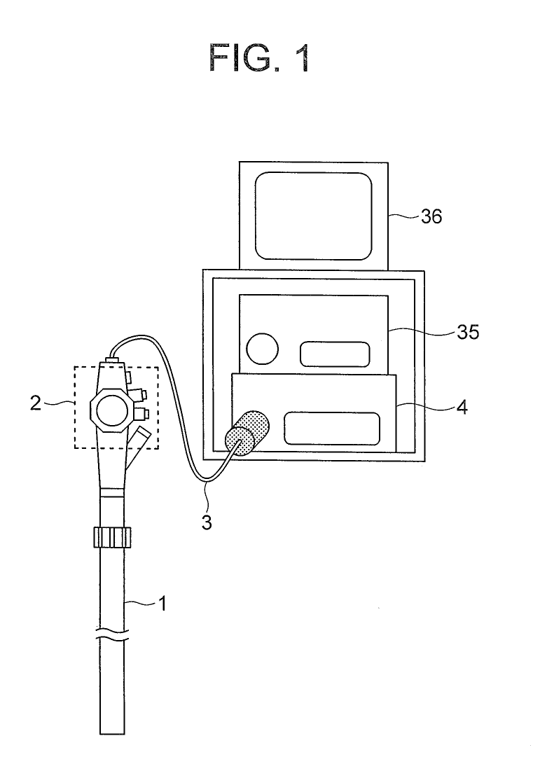

FIG. 1 is a diagram showing the construction of an endoscope apparatus equipped with an endoscope cooling apparatus according to the present invention, or an endoscope system. The endoscope system will be described with reference to FIG. 1.

The endoscope system is an observation apparatus used to observe the interior of the body of an object to be examined. An endoscope 1 is an apparatus to be inserted into the body (not shown in Figures) and has means for capturing images in the interior of the body, means for picking up living cells, and means for performing medical treatment.

An endoscope operation part 2 is adapted to be held by a user of the endoscope and provided with a mechanism for adjusting the direction of the end of the endoscope 1. The endoscope 1 is electrically and mechanically connected with a universal cord 3, a light source device 4, a video processor 35, and a monitor 36, which function in the manners described in the following.

The universal cord 3 is a cord connecting the endoscope operation part 2 and the light source device 4. The universal cord 3 is a component in which a lot of wires providing electrical and mechanical connections are arranged. The light source device 4 is a device that drives light emitted from the end of the endoscope 1. The video processor 5 is a device that performs processing of images transmitted from the endoscope 1 and synchronization and processing of circuits. The monitor 6 is used to display images captured by the endoscope 1.

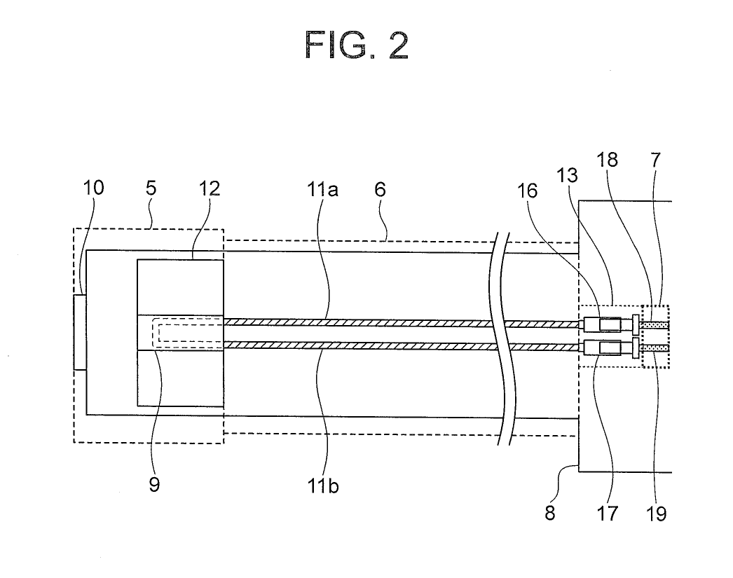

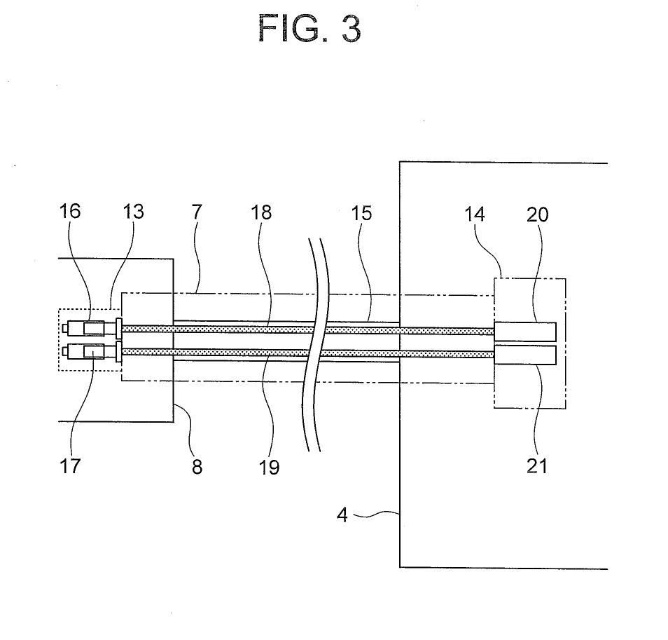

FIGS. 2 and 3 illustrate the construction of a first embodiment of the endoscope cooling apparatus according to the present invention. FIG. 2 is a cross sectional view showing the construction of the endoscope 1. FIG. 3 illustrates the connection of the endoscope 1 and the light source device 4. In the following, how the end of the endoscope is cooled will be described with reference to FIGS. 2 and 3.

The endoscope system is basically composed of an end part (rigid end part) 5, a shaft part 6, an operation part 8, a universal cord 15, and a light source device 4. A cooling part for cooling the end portion of the endoscope 1 is provided in these components. The end part 5 is provided with an illumination window 10 for illuminating light against an object to be observed.

The cooling part is made up of three cooperating portions, which include a portion that provides cooling, a portion that generates an external force for driving the cooling part, and a portion that transmits the external force to the cooling part. The portion that provides cooling is composed of a heat exchanger 9 provided in the end part 5, first and second tubes 11a, 11b provided in the shaft part 6, and a displacement-to-flow converting part 13 provided in the operation part 8.

The portion that generates an external force for driving the cooling part is composed of a displacement generating part 14 provided in the light source device 4. The portion that transmits the external force to the cooling part is composed of a displacement transmission part 7 that connects the operation part 8 and the light source device 4.

The displacement-to-flow converting part 13 is composed of syringes 16, 17. The displacement generating part 14 is composed of actuators 20, 21. The displacement transmission part 7 is composed of sheathed wires 18, 19.

Now, a water cooling system will be described. An endoscope end member 12 is arranged at the end of the endoscope 1 and houses operational components that are driven when an object to be examined is observed with the endoscope 1. The components housed therein includes, for example, an image pickup element and a light source.

The image pickup element and the light source housed therein generate heat when in operation. The heat thus generated is conducted to the endoscope end member 12 to raise the temperature of the endoscope end member 12.

In this embodiment, a system for liquid-cooling the endoscope end member 12 thus heated is provided. A heat exchanger 9 is provided in the endoscope end member 12 for the purpose of cooling. The heat exchanger 9 has a flow passage provided therein, through which cooling liquid flows. The cooling liquid is introduced in the second syringe 17.

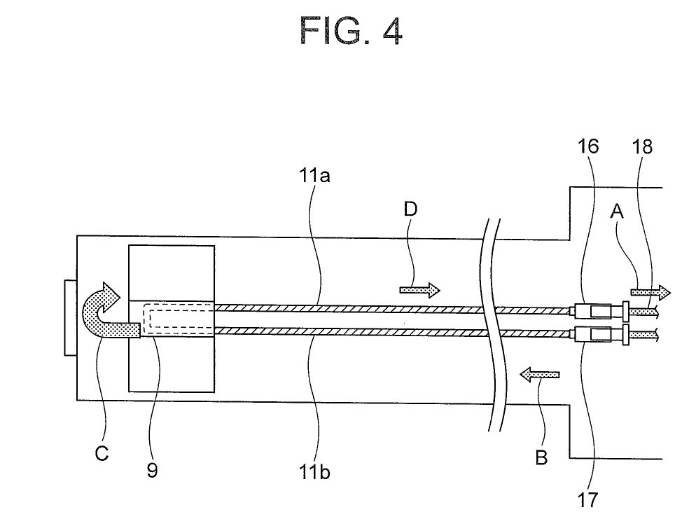

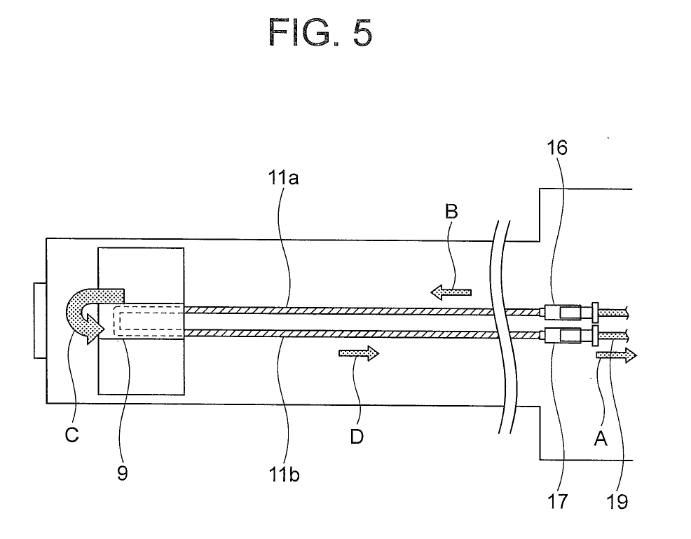

FIGS. 4 and 5 illustrate the flow of the cooling liquid.

The first sheathed wire 18 is pulled by the actuator 20 (see FIG. 3) that constitutes the displacement generating part 14. Consequently, the piston of the first syringe 16 that constitutes the displacement-to-flow converting part 13 is pulled in the direction indicated by arrow A in FIG. 4. This drives the cooling liquid in the second syringe 17 in the direction indicated by arrow B in FIG. 4. The cooling liquid moves or flows from the second syringe 17 to the second tube 11b, then to the heat exchanger 9, then to the first tube 11a, and then to the first syringe 16.

The cooling liquid undergoes heat exchange as it passes through the heat exchanger 9 in the direction indicated by arrow C in FIG. 4. Since the heat of the endoscope end member 12 is conducted to the heat exchanger 9, the cooling liquid cools the endoscope end member 12 indirectly.

The temperature of the cooling liquid is raised by the heat exchange with the endoscope end member 12. As the cooling liquid flows in the first tube 11a in the direction indicated by arrow D in FIG. 4, heat is radiated to the environment around the first tube 11a. This causes the temperature of the cooling liquid to fall. The temperature of the cooling liquid falls down to the ambient temperature by the time when the cooling liquid reaches the first syringe 16.

Next, the second sheathed wire 19 is pulled by the actuator 21, whereby the piston of the second syringe 17 is pulled in the direction indicated by arrow A in FIG. 5. The cooling liquid flows, in the direction reverse to the above-described flow, from the first syringe 16, to the first tube 11a, then to the heat exchanger 9, then to the second tube 11b, and then to the second syringe 17 to cool the endoscope end member 12 again.

In the first embodiment, the endoscope end member 12 is cooled by the to-and-fro motion of the cooling liquid. Control of the to-and-fro motion of the cooling liquid is performed by the displacement generating part 14 provided in the light source device 4, which is an external device. Therefore, cooling of the endoscope end member 12 can be achieved without a large increase in the weight of the operation part of the endoscope.

The actuators 20, 21 may be adapted to generate a pushing force instead of a pulling force.

### Second Embodiment

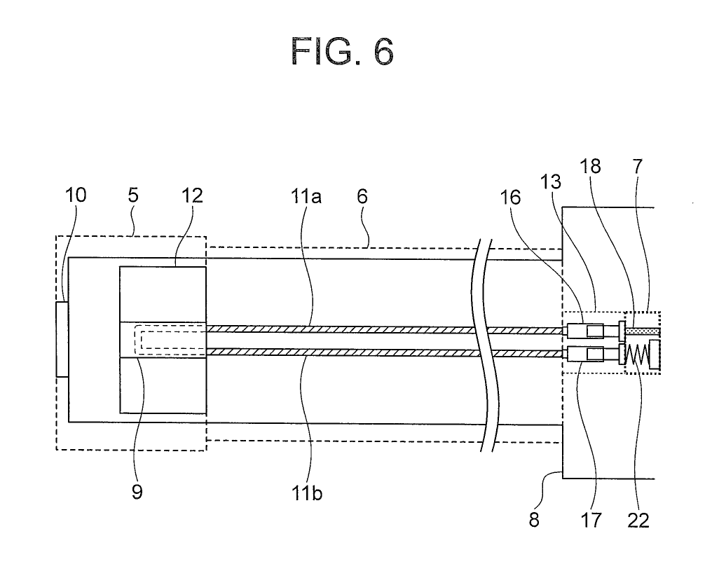

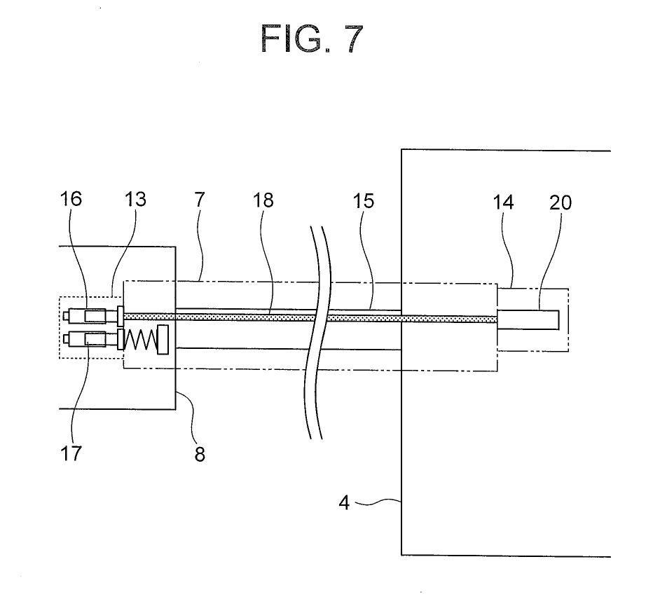

A second embodiment will be described with reference to FIGS. 6 and 7. In the second embodiment, a first sheathed wire 18 and a biasing spring 29 are used as the displacement transmission part 7. The displacement generating part 14 is composed of only an actuator 20.

Firstly, the first sheathed wire 18 is pulled by the actuator 20 in a manner similar to the first embodiment, whereby the piston of the first syringe 16 is pulled. In consequence, the cooling liquid moves or flows from the second syringe 17 to the second tube 11b, then to the heat exchanger 9, then to the first tube 11a, and then to the first syringe 16.

As the operation of the actuator 20 stops, the biasing spring 29 exerts a force that drives the cooling liquid in the reverse direction. With the driving of the cooling liquid in opposite directions, to-and-fro motion of the cooling liquid is achieved, whereby the endoscope end member 12 is cooled.

In the second embodiment, since the number of actuators 20 is smaller than that in the first embodiment by one, the overall cost of the system can be reduced. Furthermore, the electric wiring for the actuator 20 can be reduced in half. Moreover, control of the actuator 20 can be made simpler as compared to that in the case where two actuators are used. The actuator 20 in the second embodiment maybe adapted to generate a pushing force instead of a pulling force.

### Third Embodiment

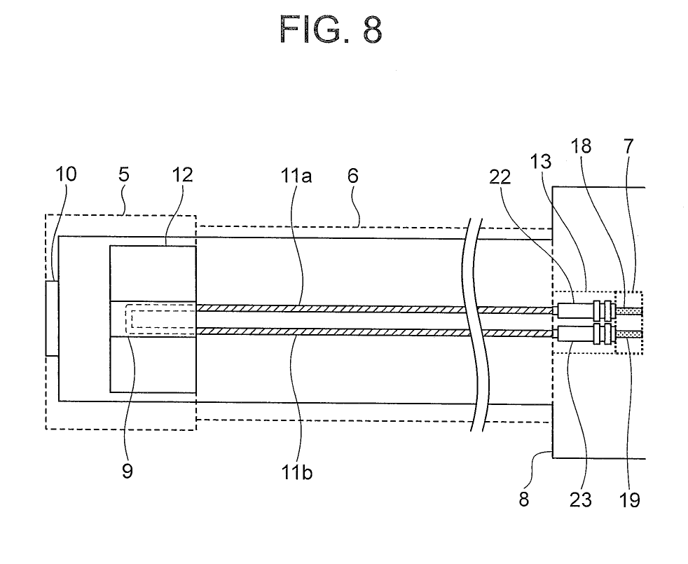

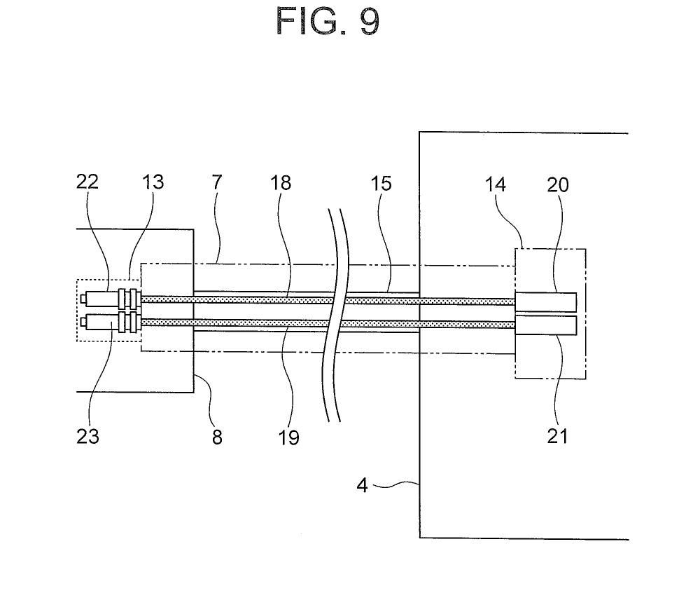

A third embodiment will be described with reference to FIGS. 8 and 9. In the third embodiment, chambers 22, 23 are used as the displacement-to-flow converting part 13, in place of the syringes 16, 17 in the first embodiment. The chambers 22, 23 have a bellows part on its back side. The operation of the bellows part exerts a force to the cooling liquid in the chambers 22, 23 to drive the cooling liquid.

In this embodiment, the displacement-to-flow converting part 13 does not have a sliding part, unlike with the first and second embodiments. Therefore, deterioration of the displacement-to-flow converting part with the long time use of the system and leakage of the cooling liquid with deterioration can be avoided.

If leakage of the cooling liquid occurs in the cooling part of the endoscope, it is not easy to fix it. Therefore, the displacement-to-flow converting part 13 having a construction not getting deteriorated greatly is very significant.

The way of driving the cooling liquid is the same as that in the first embodiment and will not be described to avoid redundancy.

### Fourth Embodiment

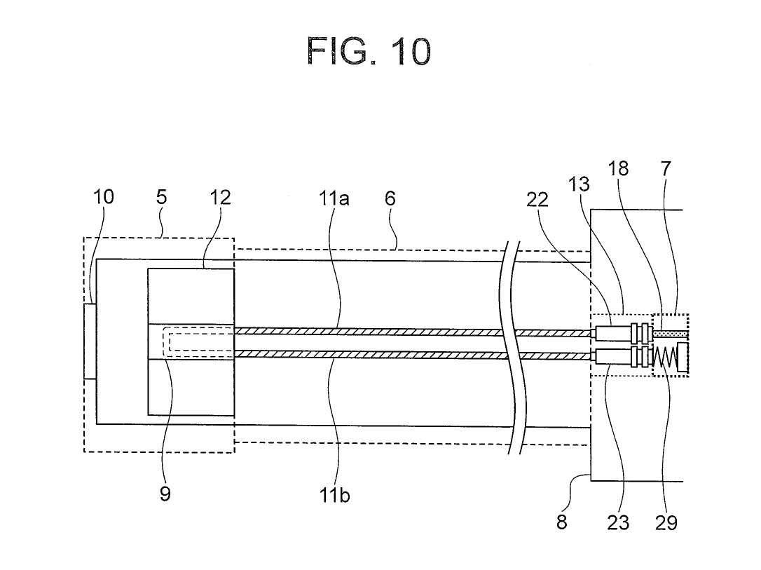

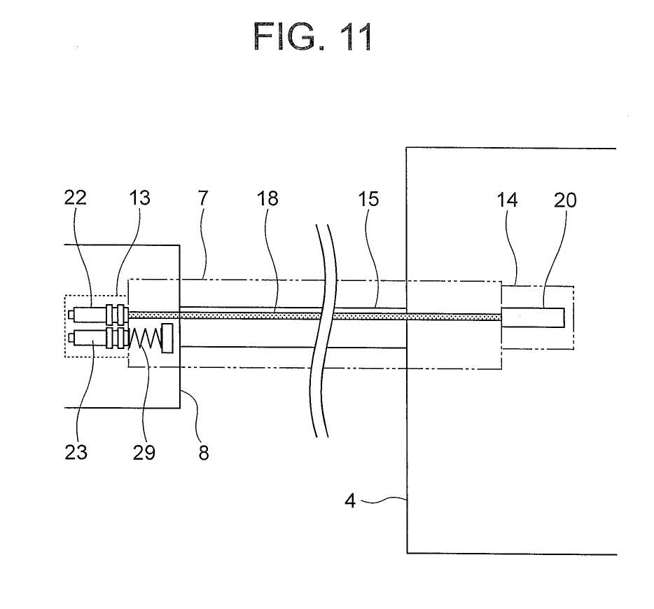

A fourth embodiment will be described with reference to FIGS. 10 and 11. In the fourth embodiment, a biasing spring 29 is used as the displacement-to-flow converting part 13 instead of a chamber. The way of driving the cooling liquid is the same as that in the second embodiment.

In this embodiment, as with in the second embodiment, the number of the actuators used to drive the displacement-to-flow converting part 13 is reduced to one, allowing a reduction in the overall cost of the system. Moreover, the electric wiring for the actuator 20 can be reduced in half, and control of the actuator 20 can be made simpler as compared to that in the case where two actuators are used.

### Fifth Embodiment

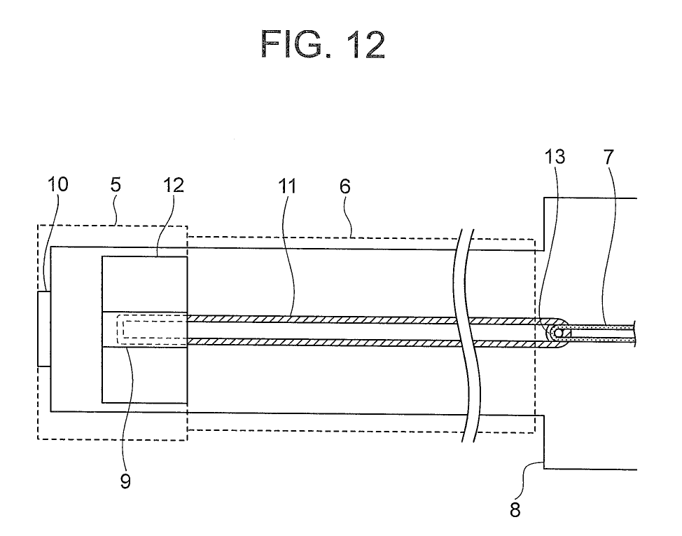

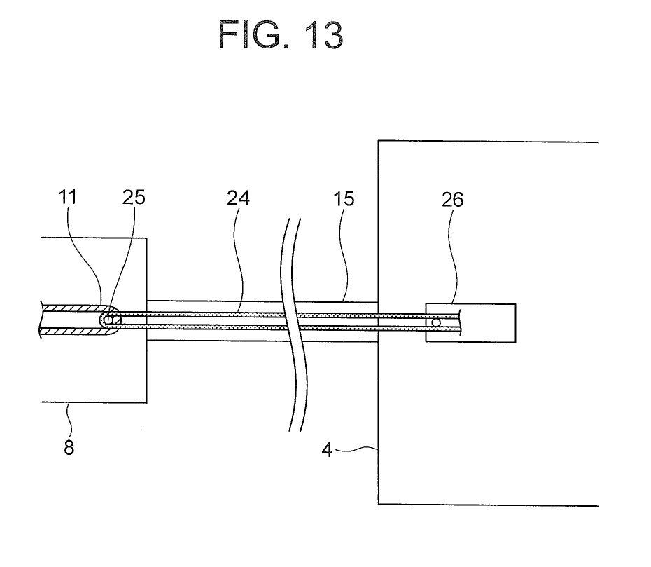

A fifth embodiment will be described with reference to FIGS. 12 and 13. In the fifth embodiment, a torque rope 24 is used in the displacement transmission part 7, and a pulley 25 is used in the displacement-to-flow converting part 13. The displacement generating part 14 is composed of a rotational force generating part 26. A tube 11 is adapted to allow cooling liquid in it not to move to and fro but to circulate.

The torque rope 24 is driven by the rotational force generating part 26, and the rotational force is converted into a horizontal force by the pulley 25 and transmitted to the tube 11 to cause the cooling liquid to circulate. When the direction of flow of the cooling liquid is to be reversed, the direction of the rotational force generated by the rotational force generating part 26 is reversed.

### Sixth Embodiment

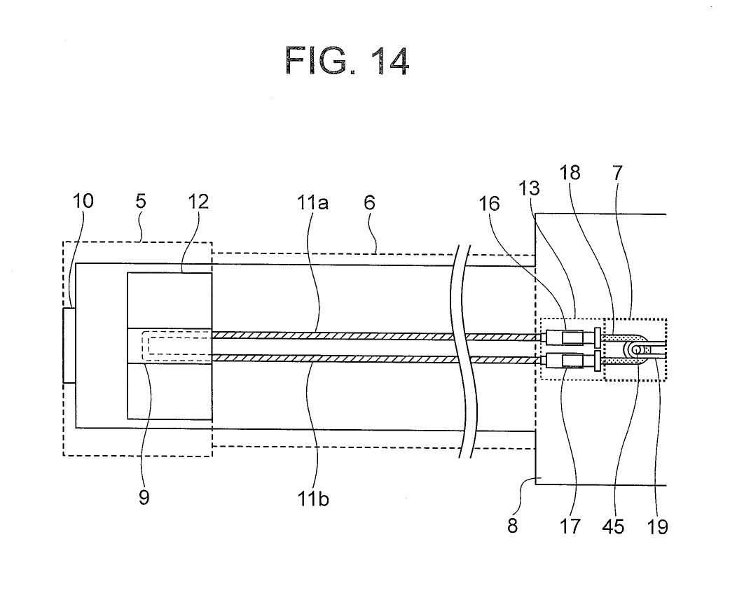

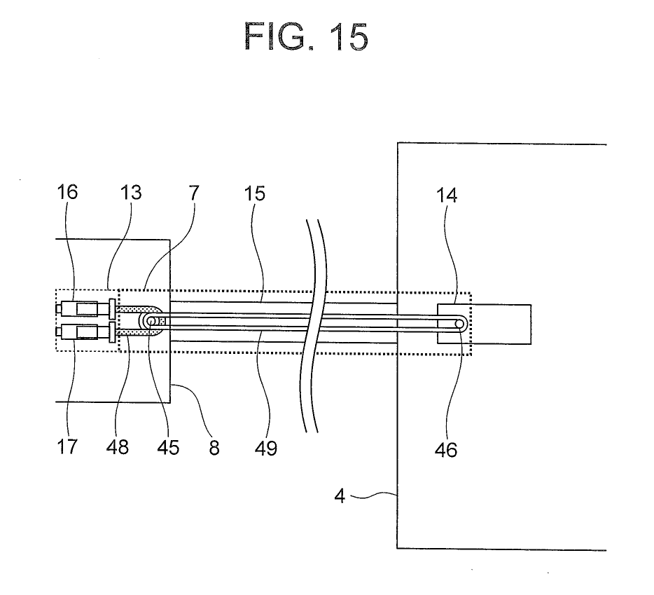

A sixth embodiment will be described with reference to FIGS. 14 and 15. In the sixth embodiment, the displacement transmitting part 7 is composed of a first wire 48, a second wire 49, and pulleys 45 and 46.

The pulley 46 and the second wire 49 are driven by the displacement generating part 14. The second wire 49 is driven to move along the longitudinal direction of the endoscope. As the second wire 49 moves, the pulley 45 connected with the second wire 49 rotates. The pulley 45 has two pulleys having different diameters integrated together and drives the first wire 48. The first wire 48 acts on syringes 16, 17 to cause the cooling liquid to move.

The driving direction of the cooling liquid can be changed by reversing the direction of displacement generated by the displacement generating part 14. Therefore, the cooling liquid can be moved to and fro.

With the above-described arrangement, cooling of the end part of the endoscope can be achieved without a large increase in the weight of the endoscope operation part 8.

### Seventh Embodiment

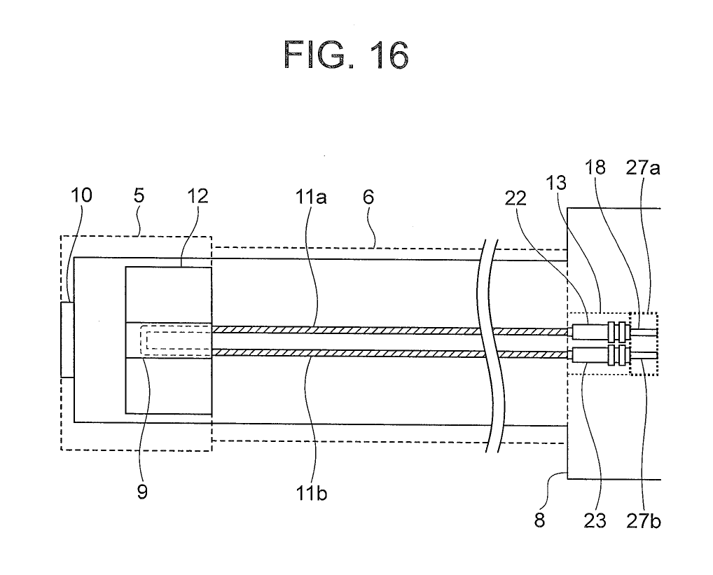

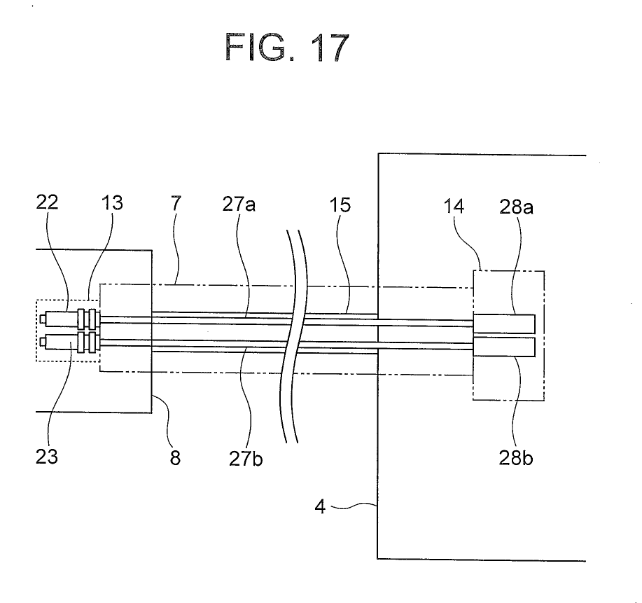

A seventh embodiment will be described with reference to FIGS. 16 and 17. In the seventh embodiment, pumps 28a, 28b are used as the displacement generating part 14 in place of the actuators 20, 21 in the third embodiment. Moreover, tubes 27a, 27b (third tube 27a, fourth tube 27b) for supplying air are used as the displacement transmission part 7 in place of the sheathed wires 18, 19 in the third embodiment.

Firstly, the first pump 28a pumps air to transmit displacement to the first chamber 22. Subsequently, the cooling liquid in the first chamber 22 is moved to the second chamber 23 in the same manner as in the third embodiment, whereby cooling is performed.

In transmitting displacement to the chambers 22, 23, transmitting an external force by air pumping can reduce friction between components better than physically exerting an external force by the sheathed wires 18, 19 or the like. Moreover, air pumping tends to apply a force to the chambers 22, 23 more uniformly.

A biasing spring like the biasing spring 29 used in the second and fourth embodiments may be used as a part that transmits a displacement to the second chamber 23 instead of the second pump 28b and the fourth tube 27b, although not shown in FIGS. 16 and 17.

With the above-described arrangement, cooling of the end part of the endoscope 1 can be achieved without a large increase in the weight of the endoscope operation part 8.

Here, an example of the above-described embodiment will be described together with specific dimensions of the components. By way of example, there will be described a case in which only such a portion of the cooling liquid sealed between the end of the endoscope 1 and the operation part 8 that is in a distance range of 30 cm from the end of the endoscope 1 is driven for cooling.

In this case, the inner diameter of the first and second tubes 11a, 11b is designed to be 0.5 mm and the inner diameter of the first and second syringes 16, 17 is designed to be 5 mm. To drive the cooling liquid in the syringes 16, 17, it is necessary that the pistons in the syringes 16, 17 be pushed/pulled to move over a distance of 3 mm. Consequently, it is necessary for the actuator 20 for driving the syringes 16, 17 to have a stroke of 3 mm. If it is assumed that the flow rate of the cooling liquid is 1 ml/min, it is necessary to change the direction of driving of the cooling liquid at intervals of 3.5 seconds.

### INDUSTRIAL APPLICABILITY

As described above, the endoscope cooling apparatus according to the present invention is useful for an endoscope system that needs cooling of the endoscope end part. In particular, it will be suitably applied to an endoscope system in which an endoscope operation part needs to be held by a hand during treatment.

### REFERENCE SIGNS LIST

1: endoscope

2, 8; endoscope operation part

3, 15: universal cord

4: light source device

5: end part

6: shaft part

7: displacement transmission part

9: heat exchanger

10: illumination window

11, 27: tube

12: endoscope end member

13: displacement-to-flow converting part

14: displacement generating part

16, 17: syringe

18, 19: sheathed wire

20, 21: actuator

22, 23: chamber

24: torque rope

25, 45, 46: pulley

26: rotational force generating part

28: pump

29: biasing spring

35: video processor

36: monitor

48, 49: wire

1. An endoscope apparatus having a scope part including an end part, a shaft part, and an operation part to be held, and an external device, the scope part comprising:
a heat exchanger provided in the end part having a flow passage for a cooling liquid for cooling a functional element that generates heat;
a first tube having a first end connected to a first end of the flow passage of the heat exchanger, and extending toward the shaft part;
a second tube having a first end connected to a second end of the flow passage of the heat exchanger, and extending toward the shaft part; and
a displacement-to-flow converting part connected to a second end of the first tube and a second end of the second tube to convert a displacement of a predetermined part to a flow of the cooling liquid,
wherein a displacement generating part is provided in the external device to generate a displacement, and
wherein the predetermined part of the displacement-to-flow converting part and the displacement generating part are connected by a displacement transmission part.

2. The endoscope cooling apparatus according to claim 1, wherein the displacement generating part generates a rotational displacement, the displacement transmission part transmits the rotational displacement, and the displacement-to-flow converting part is connected to the second end of the first tube and the second end of the second tube to cause the cooling liquid to circulate in the first tube and the second tube in response to the rotational displacement of the predetermined part.

3. The endoscope cooling apparatus according to claim 1, wherein the displacement-to-flow converting part is provided at each of the second end of the first tube and the second end of the second tube, and the cooling liquid moves in response to the displacement of the predetermined part.

4. The endoscope cooling apparatus according to claim 1, wherein the displacement-to-flow converting part performs an operation of generating a flow of the cooling liquid in the direction from the second end of the first tube to the first end of the first tube and an operation of generating a flow of the cooling liquid in the direction from the second end of the second tube to the first end of the second tube alternately and repeatedly.

5. The endoscope cooling apparatus according to claim 1, wherein the displacement generating part provided in the external device comprises a translational displacement generator, and the displacement transmission part causes the predetermined part of at least one displacement-to-flow converting part to displace translationally.

6. The endoscope cooling apparatus according to claim 5, comprising:
a first displacement-to-flow converting part provided at the second end of the first tube;
a first translational displacement generator provided in the external device and connected to the first displacement-to-flow converting part by means of the displacement transmission part;
a second displacement-to-flow converting part provided at the second end of the second tube; and
a second translational displacement generator provided in the external device and connected to the second displacement-to-flow converting part by means of the displacement transmission part,
wherein the displacement generating part alternately causes the first translational displacement generator and the second translational displacement generator to generate a pulling force, thereby generating a flow of the cooling liquid.

7. The endoscope cooling apparatus according to claim 5, comprising:
a first displacement-to-flow converting part provided at the second end of the first tube;
a first translational displacement generator provided in the external device and connected to the first displacement-to-flow converting part by means of the displacement transmission part;
a second displacement-to-flow converting part provided at the second end of the second tube; and
a biasing spring provided in a predetermined portion of the second displacement-to-flow converting part,
wherein one of forward and backward operations is carried out by a displacement of the first translational displacement generator, and the other of the forward and backward operations is carried out by a displacement caused by a stress of the biasing spring.

8. The endoscope cooling apparatus according to claim 5, wherein the displacement-to-flow converting part comprises a chamber having a volumetric capacity varying in response to the displacement of the predetermined part and filled with the cooling liquid.

9. The endoscope cooling apparatus according to claim 5, wherein the displacement generating part that generates the translational displacement comprises an actuator that generates a pulling force toward the rear end of the shaft part with respect to the longitudinal direction, the displacement-to-flow converting part comprises a first chamber connected to the actuator and having a volumetric capacity varying in response to the displacement of the predetermined part and a second chamber connected to a biasing spring and having a volumetric capacity varying in response to the displacement of the predetermined part, and the displacement-to-flow converting part causes the cooling liquid to move to and fro in the first tube and the second tube with to-and-fro motion of the actuator along the longitudinal direction of the shaft part.

10. The endoscope cooling apparatus according to claim 2, wherein the displacement-to-flow converting part is provided at each of the second end of the first tube and the second end of the second tube, and the cooling liquid moves in response to the displacement of the predetermined part.
